(19) United States
(12) Patent Application Publication
(10) Pub. No.: US 2008/0208210 A1
(43) Pub. Date: Aug. 28, 2008

(54) Uterine Manipulator

(76) Inventors: Kerry Blair, Overland Park, KS (US); Marc Gelnett, Houston, TX (US)

Correspondence Address: LATHROP & GAGE LC, 2345 GRAND AVENUE, SUITE 2800, KANSAS CITY, MO, 64108, US

(21) Appl. No.: 12/035,823

(22) Filed: Feb. 22, 2008

Related U.S. Application Data
(60) Provisional application No. 60/891,144, filed on Feb. 22, 2007.

(51) Int. Cl. A61B 17/42

(52) U.S. Cl. 606/119

(57) ABSTRACT

A uterine manipulator includes a snap-drum with a head including pitched slots, symmetrically disposed with respect to each other. This arrangement allows for a tip to be attached to the head in a short and unidirectional rotation.

### CROSS-REFERENCES TO RELATED APPLICATIONS

This application is related to and claims priority from U.S. Provisional Patent Application Ser. No. 60/891,144, entitled: Uterine Manipulator, filed on Feb. 22, 2007, the disclosure of which is incorporated by reference herein.

### TECHNICAL FIELD

The present disclosed subject matter is directed to medical instruments used to manipulate a uterus during laparoscopic examination or surgery a method associated therewith. In particular, the disclosed subject matter is directed to structure on a handle that allows for a movable tip to be easily joined to the handle and remain secured on the handle until disengagement of the tip from the handle is desired.

### BACKGROUND

Gynecologists frequently examine the internal pelvic organs of a patient by using a laparoscope. The medical procedures may include laparoscopic tubal occlusion and fertility studies. During such laparoscopic procedures, it is frequently necessary for the uterus to be repositioned in order to obtain an X-ray or to perform surgery.

Instruments have been developed to aid the surgical team in these procedures, and particularly to manipulate the uterus into a desired orientation. For example, the instruments and systems disclosed in commonly owned U.S. Pat. Nos. 5,409,496 and 5,540,700, the disclosures of both of these patents incorporated by reference herein, disclose reusable handles to which disposable tips are connected when use is desired. Attachment of the tips may be difficult to personnel lacking sufficient strength or dexterity in their hands and fingers.

### SUMMARY

The present disclosed subject matter provides an improved system for mounting a tip, such as a disposable tip, to a handle. The system is suitable for use in laparoscopies procedures as well as chromopertubation. The system includes a snap-drum at the end of a movable end of the handle. The snap-drum allows for the easy attachment of a tip by a single directional (unidirectional) short rotation or twist (through an arc of approximately 180°), free of multiple twists and turns. The snap-drum is constructed to allow for an easy and secure attachment of the tip, including by operators with minimal strength and/or dexterity. Also, this construction allows for those other than highly trained personnel to attach tips to handles, such that upon attachment, the tip will be properly oriented on the handle for proper use. This saves time, as the next procedure can begin immediately, once the tip is secured on the handle.

The disclosed subject matter is directed to an apparatus for uterine manipulation. The apparatus includes a handle unit including a proximal end and a distal end. The handle unit includes a movable member at a proximal end of the handle unit, and a drum at the distal end of the handle unit. The drum is operatively coupled to the movable member, such that movement of the movable member moves the drum about a predetermined range of motion. The drum also includes a body and a head extending from the body, the head configured for receiving the proximal end of a tip member, for example, a disposable tip, that is attached to and retained on the head by a substantially single directional (unidirectional) rotation.

Another embodiment of the disclosed subject matter includes an apparatus for uterine manipulation. The apparatus includes a handle unit including a proximal end and a distal end. The handle unit includes a movable member at a proximal end of the handle unit, and a drum at the distal end of the handle unit. The drum is operatively coupled to the movable member, such that movement of the movable member moves the drum about a predetermined range of motion (for example, approximately a 140° arc). The drum includes a body and a head extending from the body. The head includes a base coupled to the body, at least one fin extending from the base; and a slot corresponding to the at least one fin. The slot extends through a portion of the at least one fin and the base, and is, for example, pitched at an angle of approximately 30°-50° with respect to the vertical (a longitudinal axis extending through drum from the head to the body).

Another embodiment of the disclosed subject matter is directed to a method for attaching a tip member (or tip) to a handle unit to form a uterine manipulation apparatus. The method includes providing a handle unit including a proximal end and a distal end. The handle unit includes a movable member at a proximal end of the handle unit, and a drum (for example, a snap-drum) at the distal end of the handle unit. The drum is operatively coupled to the movable member, such that movement of the movable member moves the drum about a predetermined range of motion. The drum includes a body and a head extending from the body, and the head includes a base that is coupled with the body, at least one fin extending from the base, and a slot corresponding to the at least one fin, the slot extending through a portion of the at least one fin and the base. A tip member is provided that includes an elongated shaft including a proximal end and a distal end. The tip member also includes a boot of resilient material connected to the proximal end of the shaft, with the boot including structure defining a substantially enclosed recessed area distal to a substantially annular radially inwardly extending lip, for releasably coupling to the head of the handle unit. The tip member and handle unit, at the head, are brought into contact, such that the lip is caught by the slot. The tip member and/or the handle unit are rotated such that the lip extends over the base, whereby the tip is attached to the head of the drum.

Another embodiment is directed to an apparatus for uterine manipulation. The apparatus includes a handle unit including a proximal end and a distal end. The handle unit also includes a movable member at the proximal end, and a drum member, for example, a snap-drum, at the distal end. The drum member is operatively coupled to the movable member, such that movement of the movable member moves the drum member about a range of motion corresponding to the movement of the movable member. The drum member includes a base and least one slot extending through the base, of a width sufficient for catching a portion of a lip of a tip member when the tip member is placed into contact with the drum member.

### BRIEF DESCRIPTION OF THE DRAWINGS

Attention is now directed to the drawings, where like numerals or characters indicate corresponding or like components. In the drawings:

FIG. 1 is a right side elevational view of the uterine manipulator of the present disclosed subject matter, showing a portion of the handle containing part of the locking mechanism in section;

FIG. 2 is a top plan view thereof;

FIG. 3 is an enlarged, fragmentary side elevational view of the uterine manipulator hereof with a retractor tip mounted thereon, showing the range of angular movement of the tip in phantom;

FIG. 4 is a top plan view similar to FIG. 2 but in partial section along line 4-4 of FIG. 1, showing parts of the frame, handle and control wires in phantom;

FIG. 5 is a side elevational view similar to FIG. 1, showing the disengaged position of the locking lever in phantom;

FIG. 6 is a fragmentary view of the frame with the handle removed, showing the position of the control wires during elevation of the snap-drum;

FIG. 7 is a fragmentary view similar to FIG. 6, showing the snap-drum in a neutral orientation in alignment with the frame;

FIG. 8 is a fragmentary view similar to FIG. 7, showing the snap-drum in a depressed orientation;

FIG. 9 is a top plan view of the frame of the uterine manipulator, showing the central partition wall in phantom;

FIG. 10 is a side elevational view of the frame;

FIG. 11 is a bottom view of the frame;

FIG. 12 is a fragmentary vertical sectional view of the frame along lines 12-12 of FIG. 9;

FIG. 13 is a rear end view of the frame of the uterine manipulator of the present invention;

FIG. 14A is a side view of the snap-drum of the uterine manipulator;

FIG. 14B is a front view of the snap-drum of the uterine manipulator;

FIG. 14C is a top view of the snap-drum of the uterine manipulator;

FIG. 14D is a bottom view of the snap-drum of the uterine manipulator;

FIG. 14E is a side perspective view of the snap-drum of the uterine manipulator;

FIG. 15 is an enlarged side elevational view of the tip for mounting to the snap-drum of the uterine manipulator hereof, showing the balloon on the shaft of the tip in an inflated condition in phantom;

FIG. 16 is an enlarged top plan view of the tip of FIG. 15;

FIG. 17 is a front end elevational view of the tip hereof and shown in section through its shaft;

FIG. 18 is a cross-sectional view of the tip along line 18-18 of FIG. 17, with the shaft of the tip omitted for clarity;

FIGS. 19A and 19B are bottom perspective views of the distal end of the uterine manipulator and the tip showing the attachment process of the tip to the snap-drum;

FIGS. 19C and 19D are top perspective views of the distal end of the uterine manipulator and the tip showing the continued attachment process of the tip to the snap-drum;

FIG. 19E is a bottom perspective view of the distal end of the uterine manipulator and the tip showing the continued attachment process of the tip to the snap-drum; and,

FIG. 19F is a cross-sectional view of the tip along line 19F-19F of FIG. 17, showing the snap-drum for receiving the boot of the tip thereon.

### DETAILED DESCRIPTION

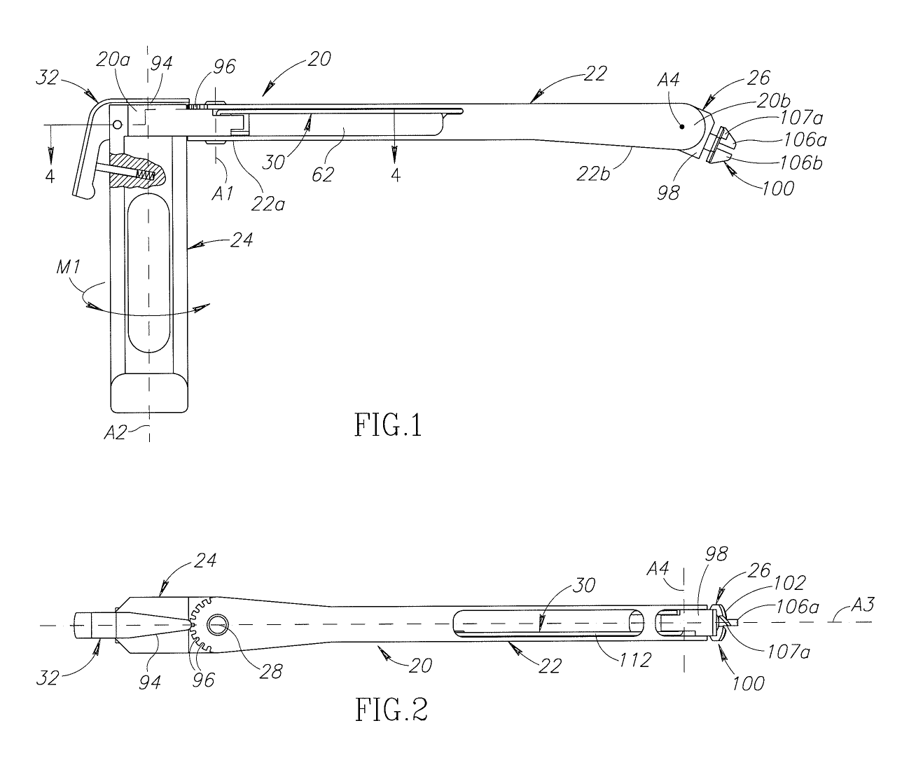

FIGS. 1-8 show a uterine manipulator 20 in accordance with the present disclosed subject matter. The uterine manipulator 20 includes an elongated frame 22, a handle 24 pivotally coupled to one end (a proximal end 22a of the frame 22, corresponding to a proximal end 20a of the uterine manipulator 20) thereof, a mounting snap-drum 26 positioned at the other end of the frame 22 (a distal end 22b of the frame 22, corresponding to a distal end 20b of the uterine manipulator 20), and, a pivot pin 28 pivotally coupling the handle 24 and the frame 22. Connecting means 30 extend along the frame 22, between the handle and snap-drum 26, for actuating movement of the snap-drum 26 through a desired angular range of motion. There is also a locking mechanism 32 for selectively locking the handle 24 against undesired pivoting relative to the frame 22. The uterine manipulator 20, except for the snap-drum 26, may be, for example, in accordance with the uterine manipulator (except the snap-drum) as disclosed in U.S. Pat. No. 5,540,700 or available commercially as the handle for the RUMI System™ Uterine Manipulator Injector, from CooperSurgical, 95 Corporate Drive, Trumbull, Conn. 06611.

In this document, references to proximal ends, sides and directions, and distal ends, sides, and directions, respectively, correspond to the proximal 20a, 22a and distal 20b, 22b ends, as established for the uterine manipulator 20 and frame 22. Additionally, in this document, references are made to directions, such as upper, lower, top, bottom, up, down, upward, downward, front, rear, above, below, left, right, lateral and variations thereof. These directional references are exemplary, to show the disclosed subject matter in an exemplary orientation, and are in no way limiting.

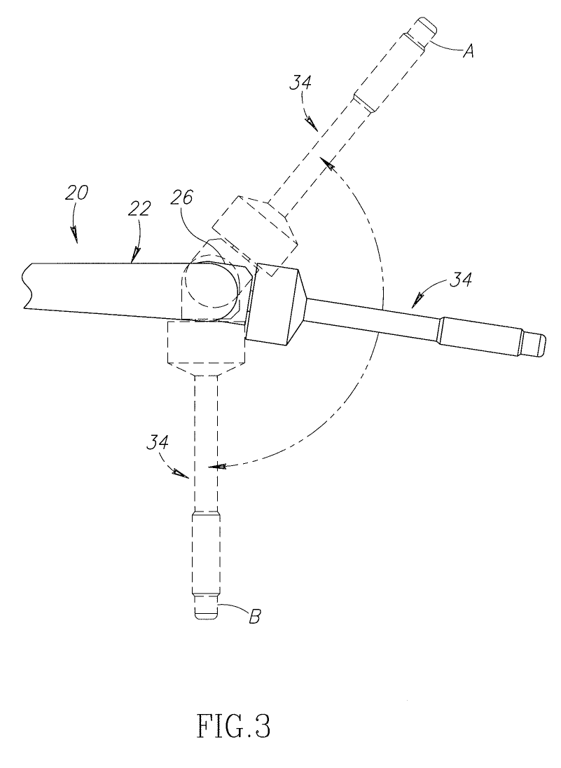

As may be seen in FIG. 3, the snap-drum 26 is shiftable through a range of motion to retrovert and antevert a removable tip 34 mounted thereon when the handle 24 is pivoted about a the pivot pin 28. The tip 34 is described in detail below, but may also be, for example, a tip as disclosed in U.S. Pat. No. 5,540,700, or a tip from a product line that is commercially available as the RUMI System™ Disposable Uterine Manipulator Tip, from CooperSurgical, 95 Corporate Drive, Trumbull, Conn. 06611.

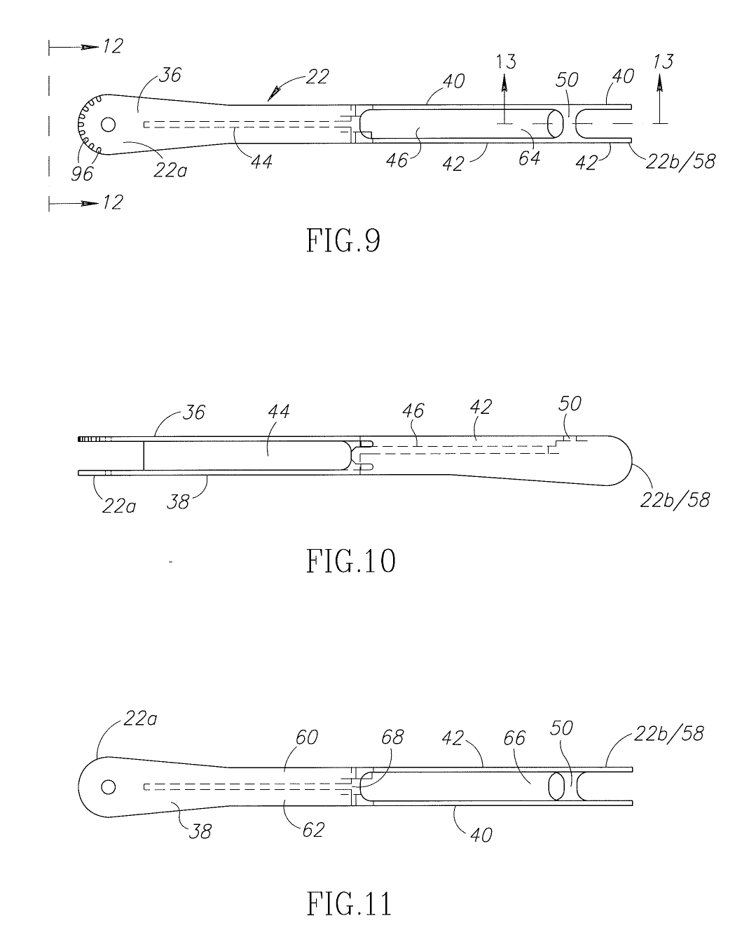

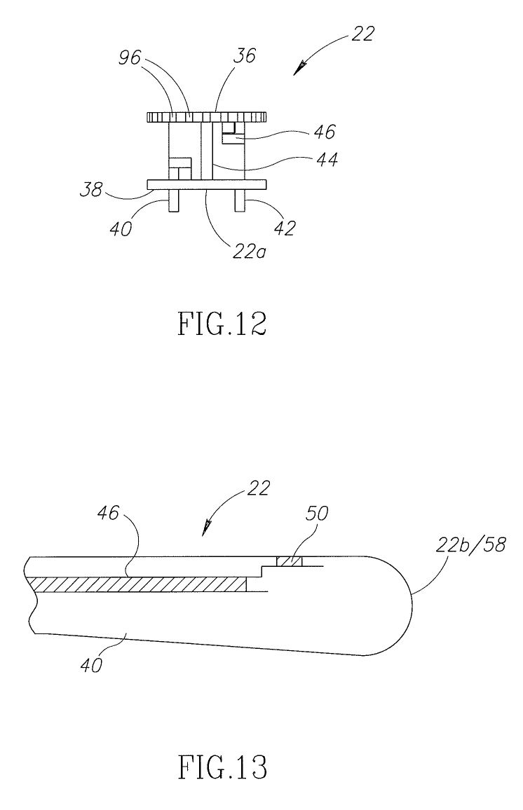

In greater detail, the frame 22 is of a modified I-beam construction as shown in FIGS. 9-13, which includes a top wall 36, a bottom wall 38, sidewalls 40 and 42, upright beam 44, transverse beam 46, and upper brace 50, which serves to restrict and constrain the connecting means 30 within the frame. The top wall 36 and bottom wall 38 are substantially flat and provided with aligned holes 52 and 54 for receiving the pivot pin 28 therein, and are wider at the proximal end 22a than the narrower distal end 22b (also known as the remote end 58) of the frame 22.

As may be seen in FIG. 10, side access ports 60 and 62 are presented by the space between the top wall 36 and the bottom wall 38 on the proximal side of the transverse beam 46. Similarly, top access port 64 and bottom access port 66 are presented by the space between the side walls 40 and 42 on the remote side of the upright beam 44. The upright beam 44 interconnects the top wall 36 and the bottom wall 38 and is joined at a cruciform junction 68 with transverse beam 46 to support the frame 22. Similarly, the transverse beam 46 and upper brace 50 interconnect the sidewalls 40 and 42, and serve to maintain the sidewalls 40 and 42 in properly spaced-apart relationship for constraining the snap-drum 26 therebetween. Sidewalls 40 and 42 present a gradually increasing vertical dimension more proximal to the distal end 22b (remote end 58), as may be seen in FIG. 10. The frame 22 is preferably cast or otherwise formed of stainless steel, that is, for example, autoclavable, although rigid synthetic resin material could also be used to provide a more economical unit for single-use applications.

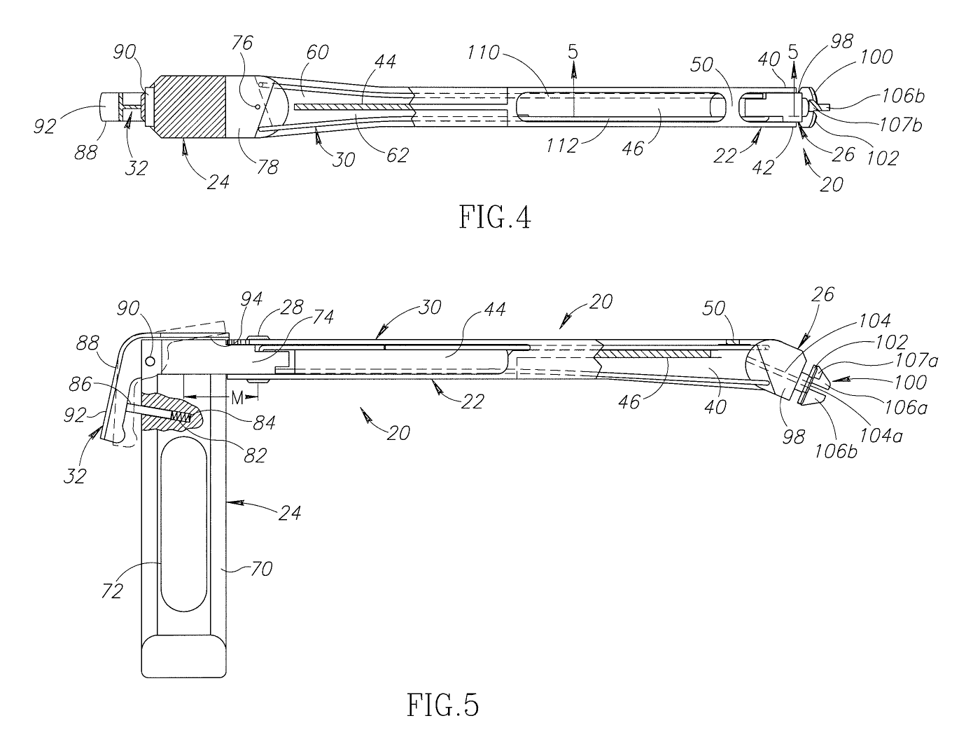

The handle 24 is preferably generally machined or alternatively molded of synthetic resin (for example, also autoclavable) in an inverted L-shape, and is provided with a graspable section 70 defining a central opening 72 which both reduces weight and enables the user to pass a length of fabric or other supporting material therethrough to suspend the handle 24 during surgery. The handle 24 further includes an arm 74 projecting from the graspable section 70 which includes an aperture 76 as seen in FIG. 4. The graspable section 70 is elongated and aligned with but offset from the aperture 76 adapted for connecting to the frame 22, thereby defining a moment arm M when the handle 24 is pivoted relative to the frame 22 (shown, for example, by the arrows M1 of FIG. 1.

The handle 24 includes a top relieved area 78 along the remote part of the arm 74 for permitting the connecting means 30 to swing through a desired range of motion, and a similar bottom relieved area 80 on the bottom side of the remote part of the arm 74. The graspable section 70 further includes a bore 82 opening toward the rear or proximal side of the uterine manipulator 20 for receiving a spring 84 and a rod 86, which serve in locking the handle 24 in the desired position relative to the frame 22.

To that end, the proximal end of the handle 24 mounts a locking pawl 88 which is part of the locking mechanism 32. The pawl 88 is pivotally connected to the handle 24 by a pin 90. The pawl 88 includes a thumb latch 92 and catch 94. The thumb latch 92 is normally biased away from the handle 24 by the rod 86, whereby the catch 94 engages a selected notch 96 on the proximal end 22a of the frame 22. The pawl 88 is preferably molded of synthetic resin material, and is, for example, autoclavable.

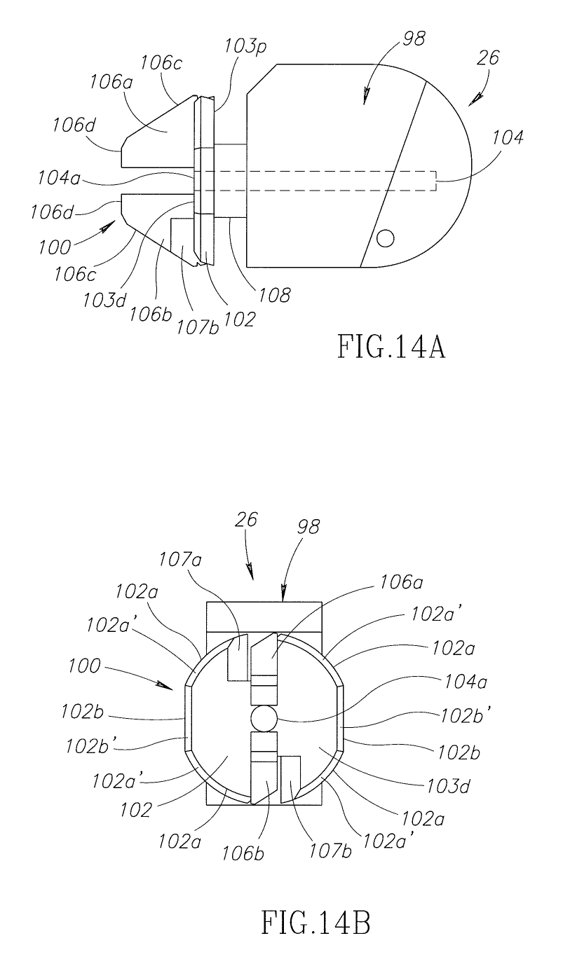

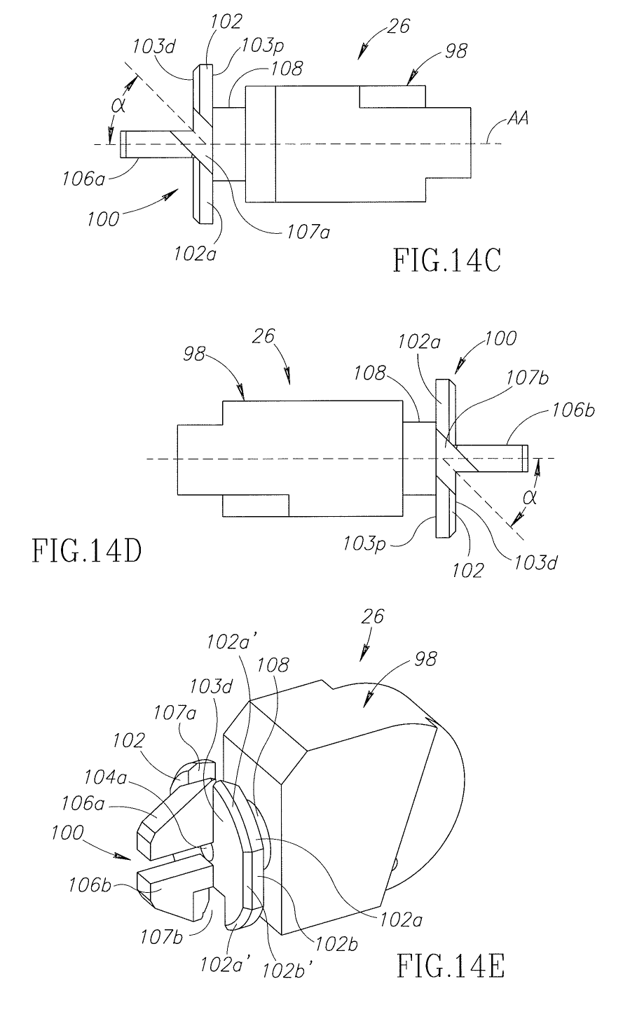

Snap-drum 26, as shown in detail in FIGS. 14A-14E, to which attention is also directed, is, for example, a unitary member, made of materials such as medical grade stainless steel that is, for example, autoclavable. The snap-drum 26 includes a flat-sided body 98 configured to fit between the sidewalls 40 and 42 of the frame 22 at the distal end 22b (remote end 58) thereof, and a head 100 which projects outwardly from the body 98. The head 100 includes a rim or base 102. The rim or base 102 has a distal side 103d and a proximal side 103p. The rim 102 extends laterally, to about the width of the external sides of the sidewalls 40 and 42. The rim 102 is formed of oppositely disposed rounded portions 102a, defining a rounded edge 102a′, and oppositely disposed straight portions 102b, with a straight edge 102b′. The edges 102a′, 102b′ are tapered or rounded outward, from the distal side 103d of the rim 102, to the proximal side 103p of the rim 102.

A channel (bore) 104 extends internally through the snap-drum 26, from an opening 104a in the rim 102. The channel 104 receives a shaft 114 (detailed further below), that extends proximally from the tip 34, therein.

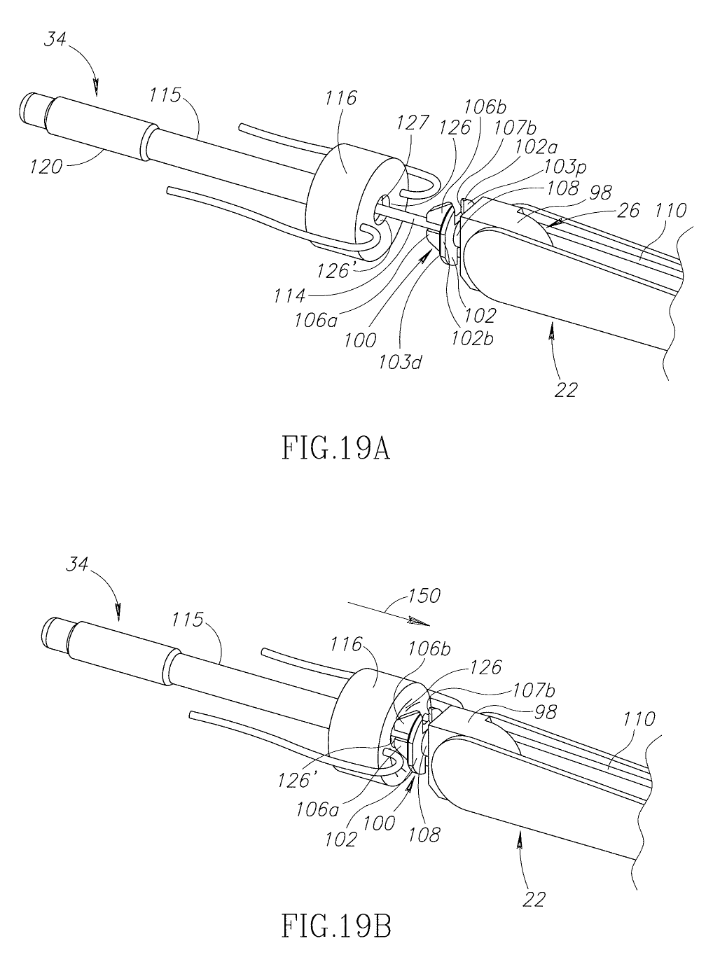

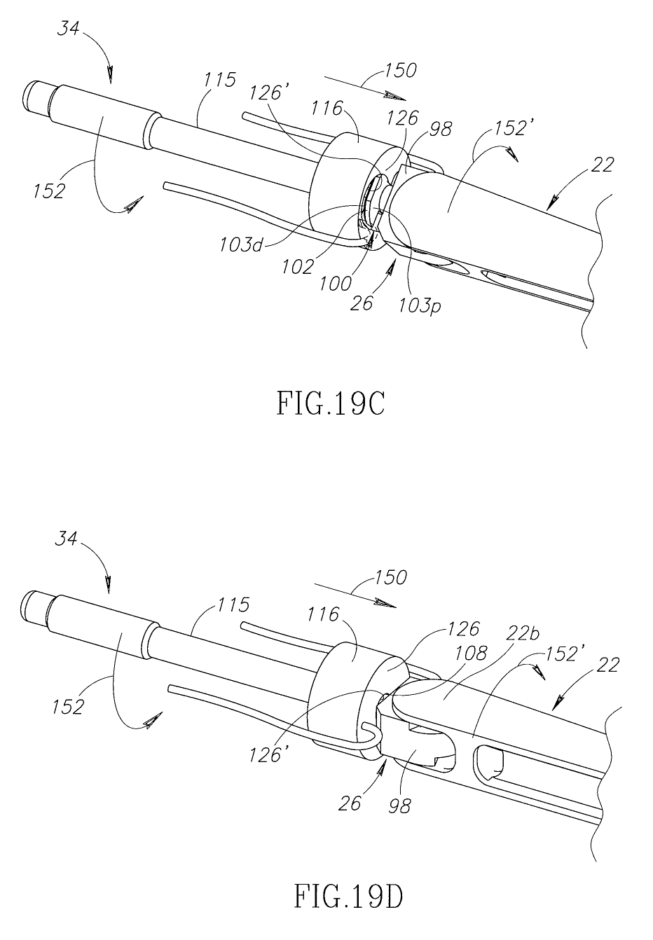

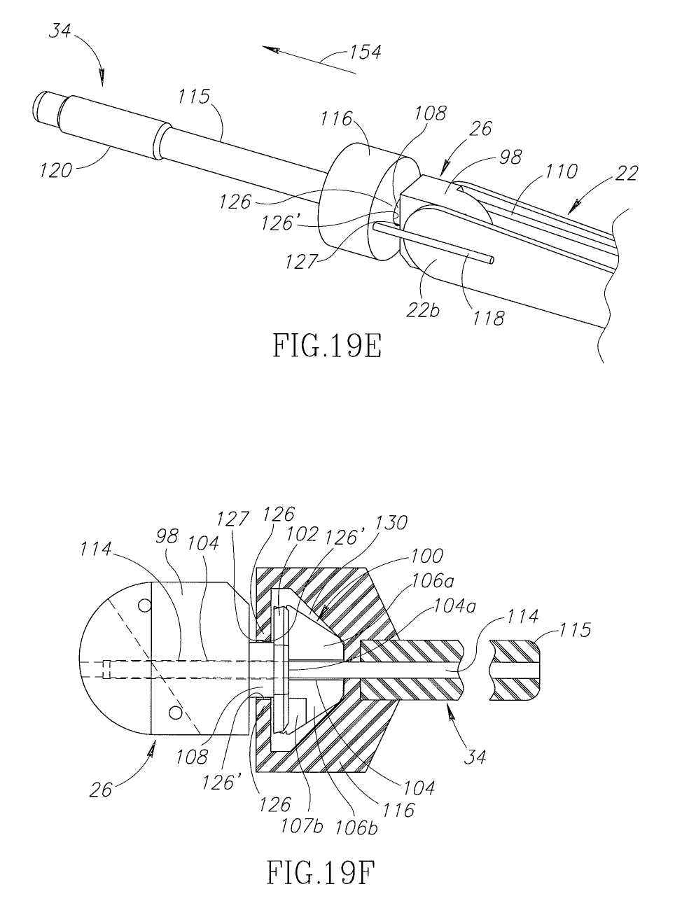

The head 100 also includes fins 106a, 106b, which project generally distally from the rim 102. The fins 106a, 106b are, for example, symmetric. The fins 106a, 106b extend, for example, along the longitudinal axis A3 and, for example, are coplanar, with a plane formed by Axis A1 and longitudinal axis A3. Each fin 106a, 106b includes an inclined portion 106c, extending from the rim 102, at its rounded edge 102a′, and terminating in a flat portion 106b, at the distal end of the head 100. Slots 107a, 107b, also symmetric as positioned on the head 100, are at opposite lateral sides of the respective fin 106a, 106b. The slots 107a, 107b run diagonally through the head 100, and are formed in rounded portion 102a of the rim 102, and a portion of the respective fin 106a, 106b. The slots 107a, 107b are uniform in width and are pitched or angled with respect to the a vertical axis AA extending through the snap-drum 26. The pitch or angle, represented by α, may be, for example, approximately 30°-50° degrees, and as a further example, approximately 45°. These slots 107a, 107b, for example, function similar to that of screw threads, as they serve to catch the lip 126 of the boot 116 at the proximal end of the tip 34, for engaging the tip 34 during, when the tip 34 is being attached to the snap-drum 26, by being rotated on to the head 100 of the snap-drum 26. A neck 108 of reduced diameter connects the body 98 to the head 100, permitting enhanced connection of the tip 34 to the head 100, as the lip 126 ultimately fits over the proximal side 103p of the rim 102, for a secure engagement of the tip 34 on the snap-drum 26 (as shown in FIG. 19D).

The pivot pin 28 is, for example, of metal such as stainless steel (for example, which is autoclavable) and may be a rivet or provided with a threaded fastener at one end to provide a secure connection between the handle 24 and the frame 22.

The handle 24 and the snap-drum 26 are connected by connecting means 30, which include a pair of wires 110 and 112. The wires 110 and 112, are, for example, made of metal, such as stainless steel, that is autoclavable.

The wires 110 and 112 are bent at the proximal and distal (remote) ends thereof to fit into corresponding holes in the arm 74 and body 98, respectively. The body 98 is provided with corresponding relieved areas on each side to permit movement of the snap-drum 26 without interference between the wires 110 and 112 and the sidewalls 40 and 42.

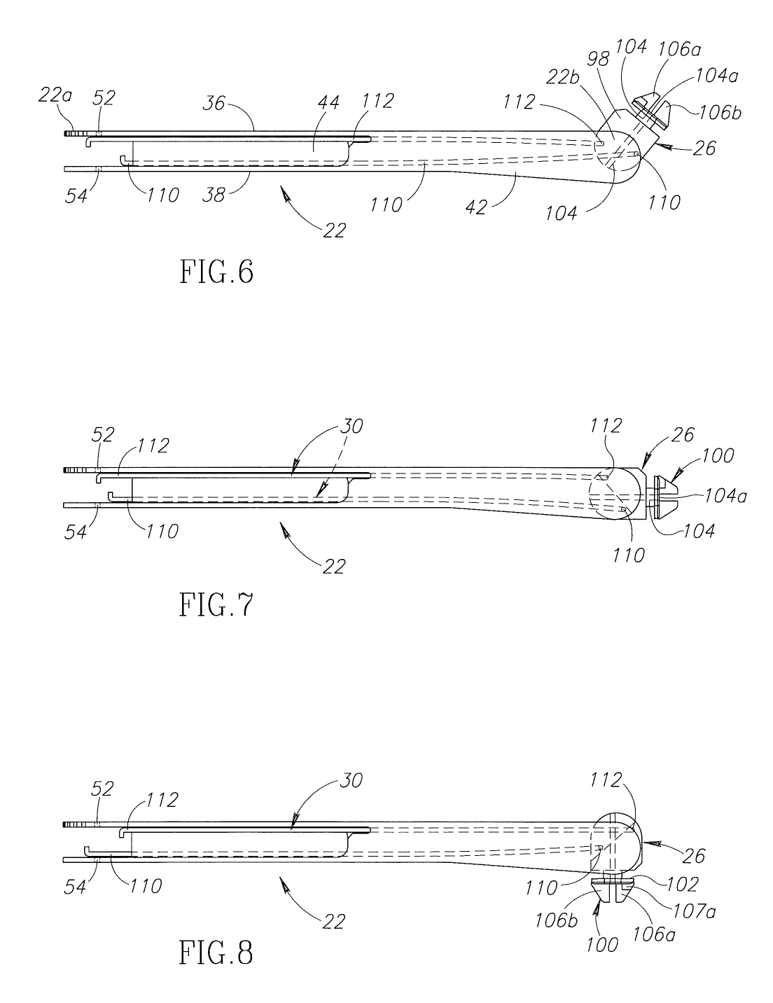

As may be seen from FIGS. 6-8, the wires 110 and 112 are shifted longitudinally to initiate corresponding motion of the snap-drum 26, this being in turn caused by pivoting of the handle 24 about an axis A1 defined by the pivot pin 28. The wire 110 extends along the length of the frame 22, whereby it is positioned above the transverse beam 46, but below the top wall 36 and the upper brace 50, and also to the left side of upright beam 44. The wire 112 extends along the length of the frame 22, and is positioned below the transverse beam 46, but above the bottom wall 38, and to the right of upright beam 44. This arrangement provides good control and stability for the wires 110 and 112 while maintaining the desired separation between the wires 110 and 112, and permits the wires 110 and 112 to cause movement of the snap-drum 26 only within the desired range of vertical motion.

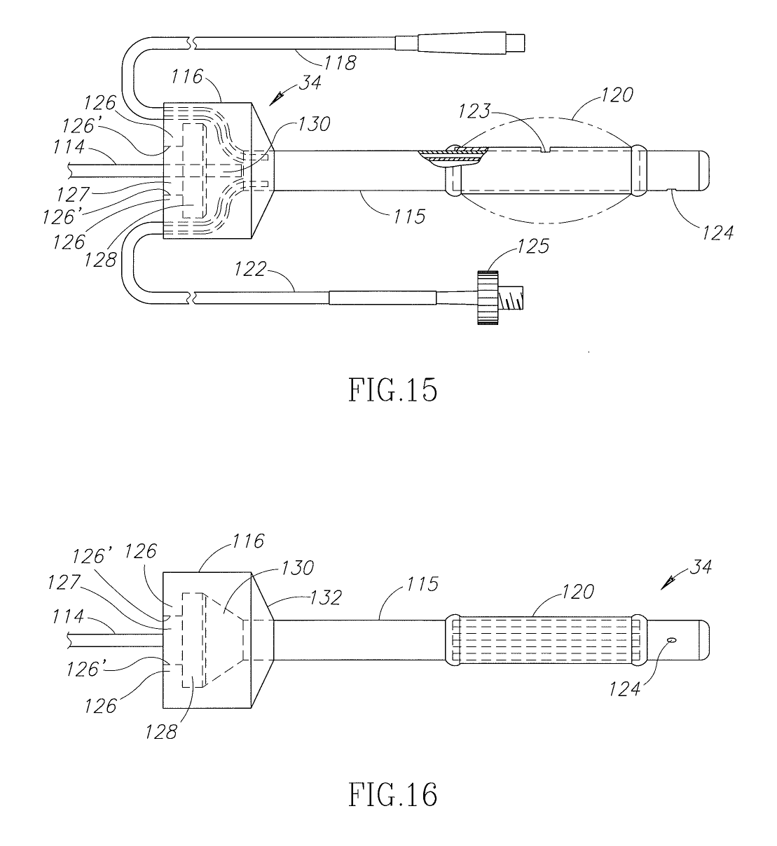

The tip 34, as shown in FIGS. 15-18, to which attention is also directed, is made of medical grade silicones (for example, USP Class IV), urethane or other flexible synthetic resin material around a rigid metal shaft 114. The shaft 114 extends normally the length of the tip 34 except for the distal or remote end of the tip 34 to permit flexibility of the distal end of the tip 34, and projects in a proximal or rearward direction to fit into the channel (bore) 104.

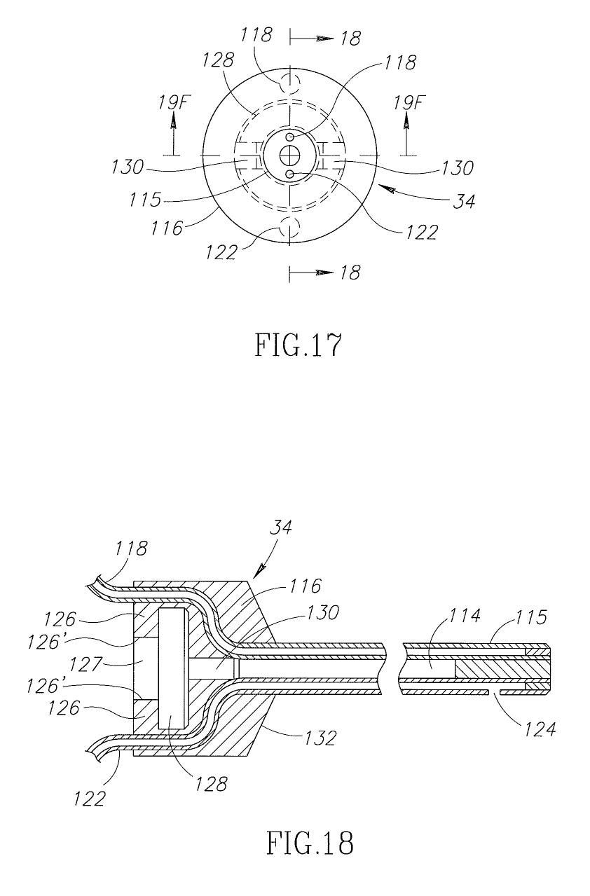

The tip 34 includes an elongated shaft 115 extending distally from a boot 116 of the aforementioned material which is circular in cross-section, as seen in FIG. 17, and receives therein balloon tube 118 which communicates air to a silicone rubber balloon 120 (shown inflated in phantom in FIG. 15), through an opening 123. The boot 116 also receives a dye tube 122 which communicates with an opening 124, defining a dye port at the remote end of the tip 34. Because a single opening could be occluded by pressure against the uterine wall during use, additional openings, serving as dye ports, oriented in different directions, to provide enhanced dye flow capability, may also be made in the shaft 115, by extending the dye tube 122 or otherwise modifying the shaft 115 by conventional techniques.

Each tube 118 and 122 is provided with the appropriate fittings to connect with respective controlled sources of pressurized air and dye. For example, the dye tube 122 at its free end may include a luer lock connector 125 for attachment to a dye syringe.

The boot 116 includes an annular radially inward projecting lip 126 (with an edge 126′), the lip 126 which presents an opening 127 to receive the head 100 of the uterine manipulator 20 therethrough, the lip 126 fitting normally proximate to the head 100 and into the neck 108. Remote to the lip 126 is a circular relieved area 128 which receives the head 100 and is complementally configured, including a remotely projecting slot or void 130 for receiving the fins 106a, 106b. The boot 116 thus effectively surrounds the head 100, the boot 116 being provided with a tapered distal or remote surface 132 for ease of insertion into the vaginal cavity.

FIGS. 19A-19F show an exemplary operation of the connection of the tip 34 to the uterine manipulator 20, at the snap-drum 26. As shown in FIG. 19A, the tip 34, at its proximal end is such that the shaft 114 is placed into the channel 104 of the snap-drum 26. The tip 34 is moved proximally (in the direction of the arrow 150), as shown in FIG. 19B, until an edge 126′ of the lip 126 moves over the fins 106a, 106b and is caught in the respective slots 107a, 107b, as shown in FIG. 19C. This proximal movement may be accompanied by a rotation of the tip 34 in the direction indicated by the arrow 152 and/or a rotation of the uterine manipulator 20 (represented by the shaft 22) in the opposite rotational direction of arrow 152′. With the lip 126 now caught, the edge 126′ slides in the slots 107a, 107b, and the aforementioned rotation(s) cause the edge 126′ to move over the proximal side 103p of the rim 102.

As shown in FIG. 19D, the pushing of the tip 34, in the direction of the arrow 150, coupled with the rotation of the tip 34 (in the direction of the arrow 152) and/or the rotation of the uterine manipulator 20 (in the direction of the arrow 152′) continues, until the as the entire lip 126 covers the proximal side 103p of the rim 102. This rotation results in the tip 34 being properly positioned (oriented) on the uterine manipulator 20, as the fins 106a, 106b, upon entering the correspondingly configured void 130, rest in accordance with the void 130 in the boot 116, the void 130 now positioned along the longitudinal axis A3, and coplanar with the plane formed by the Axis A1 and the longitudinal axis A3 (FIG. 19F). The operator may feel (tactilely sense) this entry of the fins 106a, 106b into the void 130 and their engagement therein. This causes a rest or attachment orientation for the tip 34, such that that balloon 118 and dye 122 tubes are positioned on the at the lateral sides of the shaft 22, when the tip 34 is securely attached to the snap-drum 26 (FIG. 19E).

Throughout the attachment process, the rotation is single directional, like that of a screw, as the slots 107a, 107b, act like screw threads. The pitch of the slots 107a, 107b, coupled with their location with respect to the corresponding fins 106a, 106b allows for the attachment of the tip 34 by the aforementioned single directional rotation over an arc length of approximately 180°, or an approximately half rotation. Additional rotations in the direction opposite the arrow 152 are also permissible, should minor adjustments be desired. The now attached tip 34 is shown in FIGS. 19E and 19F. The attachment can be checked for security by attempting to pull the now attached tip 34 distally, in the direction of the arrow 154, as shown in FIG. 19E.

With the tip 34 now attached to the uterine manipulator 20, the physician or operator may now connect the balloon tube 118 to a source of pressurized air (not shown), while similarly connecting the dye tube 122 to a source of medical dye (not shown) as appropriate to the examination. The tip 34 is inserted into the vaginal cavity of the patient until the tip is properly positioned in the patient's uterus. During insertion, the handle 24 may be locked in a position whereby the shaft 114 is substantially aligned with the longitudinal axis of the frame 22 by positioning the catch 94 in the desired notch 96. While the uterine manipulator 20 is shown in FIGS. 1-5 in an orientation, this is for explanation purposes, as in operative situations for uterine manipulation, the uterine manipulator 20 is normally used in an orientation inverted from that shown in FIGS. 1-5. Reference to the uterine manipulator 20 with an attached tip 34 is described in the exemplary operation below in this inverted orientation.

Once inserted, the handle 24 and proximal end of the frame 22 will project from the vaginal cavity. With the patient in a normally supine position with knees elevated, the graspable portion of the handle will be oriented upward. The physician then pivots the handle 24 relative to the frame about a pivot axis (A1) defined by the pivot pin 28, the pivot axis thus being parallel to but spaced forwardly or remote from the longitudinal axis (A2) of the graspable handle 24 and perpendicular to the longitudinal axis (A3) of the frame 22, the axes A1-A3 shown in FIGS. 1 and 2. An exemplary range of motion for the snap-drum 26 and tip 34 is approximately 140° (about an axis A4 extending through the snap-drum 26, as shown in FIGS. 1 and 2) indicated generally in FIG. 3, with pivoting of the handle 24 to the maximum clockwise position (referring to FIG. 2) reflecting position A and pivoting of the handle 24 to the maximum counterclockwise direction reflection position B. The upright beam 44 impinges on the wires 110 and alternatively 112 at the extreme ranges of motion of the handle 24 relative to the frame 22 and thereby acts as a stop or limit on the range of the tip 34 relative to the frame 22.

With the uterus properly positioned, the physician can inflate the balloon 120 to occlude the uterine passage and then inject dye or perform other functions as may be necessary. Advantageously, the handle 22 readily locks in the desired pivoted position unless the physician or operator depresses the latch 92 to release the catch 94. Thus, to reposition the tip 34, the physician or operator depresses the latch 92 on the pawl 90, pivots the handle 24 relative to the frame 22, and releases the latch 92 to lock the tip 34 into the desired orientation. After the procedure is complete, the balloon 120 is deflated, the tip 34 is positioned in substantial alignment with the longitudinal axis A3 (FIG. 2) of the frame 22, and withdrawn.

If the physician or operator's hands must be freed during the procedure, a length of gauze or other material may be looped through the central opening 72 in the handle 24, as shown in FIG. 5, and then the gauze may be tied about the patient's knees or other support to hold the uterine manipulator 20 in position.

Turning back to FIGS. 19E and 19F, the tip 34 may be disengaged from the uterine manipulator 20 by twisting it (in a rotational direction opposite of the attachment rotational direction) coupled with pulling in a distal direction (the direction of the arrow 154). The tip 34 is now disengaged from the head 100 of the snap-drum 26 and separated from the uterine manipulator 20. The tip 34 may now be discarded in accordance with local medical and/or hazardous waste procedures.

While preferred embodiments of the disclosed subject matter have been described, so as to enable one of skill in the art to practice the disclosed subject matter, the preceding description is intended to be exemplary only. It should not be used to limit the scope of the disclosed subject matter, which should be determined by reference to the following claims.

What is claimed is:

1. An apparatus for uterine manipulation comprising:
a handle unit including a proximal end and a distal end, the handle unit including:
a movable member at a proximal end of the handle unit; and
a drum at the distal end of the handle unit, the drum in communication with the movable member, such that movement of the movable member moves the drum about a predetermined range of motion, the drum including a body and a head extending from the body, the head configured for receiving the proximal end of a tip member that is attached to and retained on the head by a substantially single directional rotation.

2. The apparatus of claim 1, wherein the movable member includes a gripping portion.

3. The apparatus of claim 1, wherein the predetermined range of motion includes an arc of approximately 140°.

4. The apparatus of claim 1, wherein the head includes:
a base in communication with the body;
at least one fin extending from the base; and
a slot corresponding to the at least one fin, the slot extending through a portion of the at least one fin and the base.

5. The apparatus of claim 4, wherein the slot extends inward with respect to the drum from a side of the at least one fin through the base.

6. The apparatus of claim 5, wherein the slot extends inward so as to be pitched at an angle of approximately 30° to 50° with respect to a longitudinal axis extending through the drum from the head to the body.

7. The apparatus of claim 6, wherein the first and second slots are of a width suitable for catching and holding a lip of a tip member.

8. The apparatus of claim 7, wherein the at least one fin is triangular in shape and is inclined inward from the edge of the base.

9. The apparatus of claim 8, wherein the at least one fin and corresponding slot includes two fins, a first fin and a second fin, each of the first fin and the second fin including a corresponding slot, the first fin and corresponding slot oppositely disposed from the second fin and corresponding slot on the base.

10. The apparatus of claim 9, wherein the first fin and corresponding slot and second fin and corresponding slot are symmetrically disposed on the base.

11. The apparatus of claim 10, wherein the base includes oppositely disposed rounded portions and oppositely disposed straight portions, intermediate the oppositely disposed rounded portions.

12. The apparatus of claim 10, additionally comprising:
a tip member including;
an elongated shaft including a proximal end and a distal end; and
a boot of resilient material connected to the proximal end of the shaft, the boot including structure defining a substantially enclosed recessed area distal to a substantially annular radially inwardly extending lip for releasably coupling to the head of the handle unit.

13. The apparatus of claim 10, wherein the drum includes a bore extending from the base in the direction of the body for receiving a rod extending from a tip member.

14. The apparatus of claim 13, additionally comprising:
a tip member including;
an elongated shaft including a proximal end and a distal end;
a boot of resilient material connected to the proximal end of the shaft, the boot including structure defining a substantially enclosed recessed area distal to a substantially annular radially inwardly extending lip for releasably coupling to the head of the handle unit; and
a rod mounted in the elongated shaft so as to be coaxial therewith, the rod extending proximally from the boot for being received in the bore when the tip member is attached to the head.

15. An apparatus for uterine manipulation comprising:
a handle unit including a proximal end and a distal end, the handle unit including:
a movable member at a proximal end of the handle unit; and
a drum at the distal end of the handle unit, the drum in communication with the movable member, such that movement of the movable member moves the drum about a predetermined range of motion, the drum including a body and a head extending from the body, the head comprising:
a base in communication with the body;
at least one fin extending from the base; and
a slot corresponding to the at least one fin, the slot extending through a portion of the at least one fin and the base.

16. The apparatus of claim 15, wherein the movable member includes a gripping portion.

17. The apparatus of claim 15, wherein the predetermined range of motion includes an arc of approximately 140°.

18. The apparatus of claim 15, wherein the slot extends inward with respect to the drum from a side of the at least one fin through the base.

19. The apparatus of claim 18, wherein the slot extends inward so as to be pitched at an angle of approximately 30° to 50° with respect to a longitudinal axis extending through the drum from the head to the body.

20. The apparatus of claim 19, wherein the first and second slots are of a width suitable for catching and holding a lip of a tip member.

21. The apparatus of claim 20, wherein the at least one fin is triangular in shape and is inclined inward from the edge of the base.

22. The apparatus of claim 21, wherein the at least one fin and corresponding slot includes two fins, a first fin and a second fin, each of the first fin and the second fin including a corresponding slot, the first fin and corresponding slot oppositely disposed from the second fin and corresponding slot on the base.

23. The apparatus of claim 22, wherein the first fin and corresponding slot and second fin and corresponding slot are symmetrically disposed on the base.

24. The apparatus of claim 23, wherein the base includes oppositely disposed rounded portions and oppositely disposed straight portions, intermediate the oppositely disposed rounded portions.

25. The apparatus of claim 23, additionally comprising:
a tip member including;
an elongated shaft including a proximal end and a distal end; and
a boot of resilient material connected to the proximal end of the shaft, the boot including structure defining a substantially enclosed recessed area distal to a substantially annular radially inwardly extending lip for releasably coupling to the head of the handle unit.

26. The apparatus of claim 23, wherein the drum includes a bore extending from the base in the direction of the body for receiving a rod extending from a tip member.

27. The apparatus of claim 26, additionally comprising:
a tip member including;
an elongated shaft including a proximal end and a distal end;
a boot of resilient material connected to the proximal end of the shaft, the boot including structure defining a substantially enclosed recessed area distal to a substantially annular radially inwardly extending lip for releasably coupling to the head of the handle unit; and
a rod mounted in the elongated shaft so as to be coaxial therewith, the rod extending proximally from the boot for being received in the bore when the tip member is attached to the head.

28. The apparatus of claim 23, additionally comprising:
a tip member including;
an elongated shaft including a proximal end and a distal end; and
a boot of resilient material connected to the proximal end of the shaft, the boot including structure defining a substantially enclosed recessed area distal to a substantially annular radially inwardly extending lip for releasably coupling to the head of the handle unit, the recessed area including a portion correspondingly configured for the shape of the first and second fins, such once the tip member is attached to the head, the first and second fins rest in the portion and the tip member is oriented with respect to the handle unit.

29. A method for attaching a tip member to a handle unit to form a uterine manipulation apparatus, comprising:
providing a handle unit including a proximal end and a distal end, the handle unit comprising:
a movable member at a proximal end of the handle unit; and
a drum at the distal end of the handle unit, the drum in communication with the movable member, such that movement of the movable member moves the drum about a predetermined range of motion, the drum including a body and a head extending from the body, the head comprising:
a base in communication with the body;
at least one fin extending from the base; and
a slot corresponding to the at least one fin, the slot extending through a portion of the at least one fin and the base;
providing a tip member comprising;
an elongated shaft including a proximal end and a distal end; and
a boot of resilient material connected to the proximal end of the shaft, the boot including structure defining a substantially enclosed recessed area distal to a substantially annular radially inwardly extending lip for releasably coupling to the head of the handle unit; and,
bringing the tip member into contact with the handle unit so that the lip is caught by the slot.

30. The method of claim 29, additionally comprising:
rotating the tip member such that the lip extends over the base.

31. The method of claim 30, wherein rotating the tip member includes rotating in a substantially unidirectional rotation.

32. The method of claim 29, additionally comprising: rotating at least one of the tip member or the handle unit such that the lip extends over the base.

33. The method of claim 32, wherein rotating the tip member includes rotating in a substantially unidirectional rotation and rotating the handle unit includes rotating in a substantially unidirectional rotation.

34. An apparatus for uterine manipulation comprising:
a handle unit including a proximal end and a distal end and comprising:
a movable member at the proximal end; and
a drum member at the distal end, the drum member in communication with the movable member, such that movement of the movable member moves the drum member about a range of motion corresponding to the movement of the movable member, the drum member including a base and least one slot extending through the base configured for catching a portion of a lip of a tip member when the tip member is placed into contact with the drum.

35. The apparatus of claim 34, additionally comprising:
a tip member including;
an elongated shaft including a proximal end and a distal end; and
a boot of resilient material connected to the proximal end of the shaft, the boot including structure defining a substantially enclosed recessed area distal to a substantially annular radially inwardly extending lip for releasably coupling to the head of the drum member and for extending over the base when the tip member is attached to the drum member.

36. The apparatus of claim 34, wherein the at least one slot includes two slots disposed on opposite sides of the base and symmetric to each other.

37. The apparatus of claim 36, wherein the slots are pitched at an angle with respect to a longitudinal axis extending through the drum member.

38. The apparatus of claim 37, wherein the angle is approximately 30° to 50°.

39. The apparatus of claim 34, wherein the movable member includes a gripping portion.

40. The apparatus of claim 34, wherein the predetermined range of motion includes an arc of approximately 140°.
